(19) United States
(12) Patent Application Publication
(10) Pub. No.: US 2007/0231279 A1
(43) Pub. Date: Oct. 4, 2007

(54) Cosmetic self-tanning compositions

(75) Inventors: Jens Schulz, Schenefeld (DE); Corina Zu Putlitz, Hamburg (DE); Stephanie Von Der Fecht, Schenefeld (DE); Katja Luettig, Hamburg (DE); Rainer Kroepke, Schenefeld (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/483,674

(22) Filed: Jul. 11, 2006

(30) Foreign Application Priority Data
Apr. 3, 2006 (DE) 20 2006 005 494.4

(51) Int. Cl. A61K 8/97; A61K 36/752; A61K 36/16; A61K 36/48

(52) U.S. Cl. 424/59; 424/74; 424/736; 424/757; 424/752

(57) ABSTRACT

A cosmetic self-tanning composition which comprises dihydroxyacetone and a ginkgo extract and/or grapeseed oil and/or a lotus blossom extract. This Abstract is not intended to define the invention disclosed in the specification, nor intended to limit the scope of the invention in any way.

### CROSS-REFERENCE TO RELATED APPLICATIONS

The present application claims priority under 35 U.S.C. § 119 of German Utility Model Application No. 20 2006 005 494.4, filed Apr. 3, 2006, the entire disclosure whereof is expressly incorporated by reference herein.

### BACKGROUND OF THE INVENTION

1. Field of the Invention

The present invention relates to cosmetic self-tanning preparations which comprise dihydroxyacetone and a ginkgo extract and/or grape-seed oil and/or a lotus blossom extract.

2. Discussion of Background Information

Although natural tanning through the effect of solar radiation is the most dangerous, it is also still the most frequent and popular method of obtaining a desired pigmentation of the skin. The sun can be enjoyed and utilized virtually everywhere: during a beach holiday at the sunny coast, when skiing, when hiking in the mountains, during sports or also only during a quick sunbathe during your lunch break. Sunbathing—in moderation—is pleasantly relaxing, calming and restorative. It improves the mood and spirits. The positive effects on the entire organism through the stimulation of various metabolic processes should not be forgotten either.

However, solar radiation is also associated with a number of negative effects: The harmful effect of the ultraviolet part of solar radiation on the skin is generally known. Depending on its respective wavelength, the radiation has different effects on the skin as an organ. Accordingly, many consumers have become more careful in dealing with the sun. Their attitude to sunbathing has changed: tanning at any cost is out, healthy tanning is in.

Artificial tanning means all the methods to give the skin a tanned appearance without the effect of solar radiation. Several methods are conceivable for this.

The application of cosmetics is the simplest method. These are dye preparations that only physically adhere to the skin surface, in contrast to the self-tanning preparations that chemically bond to components of the horny layer of the epidermis.

Another method of artificial tanning is taking carotenoids in the form of capsules or coated tablets, which leads to a yellowish-brown coloring of the skin.

Furthermore, the skin can be tanned without the effect of solar radiation if it is irradiated using technically produced light sources that are able to emit ultraviolet rays of different types and strengths.

While tanning the skin with ultraviolet radiation, as occurs in the natural solar spectrum or is generated by artificial light sources, can lead to considerable skin damage if exposure is too long and a certain dose is exceeded, the application of cosmetics, self-tanning creams and tanning pills containing beta carotene can be characterized as harmless and unobjectionable. Thus, whoever wants to avoid skin damage caused by light, such as sunburn, chronic light damage or possible risk of carcinoma, but does not want to give up the fashionably desired tan, must resort to those tanning methods that do not involve the application of artificially produced or natural ultraviolet radiation. The same applies to people who must not be exposed to ultraviolet because of pathological skin reactions.

The fact that some substances undergo a chemical color reaction with the keratin of (animal) skin, hair and wool, is already known from textile chemistry and vat dyeing. Recently, a class of active ingredients has become established here for cosmetic application which meets the requirements for modern cosmetic means. These active ingredients are non-irritating, are easy to use and the resulting shade is light-fast and does not wash off.

Substances with adjacent ketone and alcohol or aldehyde and alcohol groups are used, i.e., ketols or aldols that belong chiefly to the class of sugars. The most important base material still most frequently used is dihydroxyacetone (DHA), a trivalent sugar occurring in the human body. DHA reacts with the proteins and amino acids of the horny layer of the epidermis, whereby the amino group of the amino acids reacts with the keto group of the DHA in a Maillard reaction (Schiff's base). The shade is produced via a polymerization, the individual reaction steps of which have not yet been completely clarified.

This type of tanning is a purely chemical reaction of the horny layer of the epidermis and has nothing to do with a formation of new melanin. The dyed products occurring with DHA on the keratin are called melanoids. Tests have shown that the tanning properties of DHA can be strongly influenced by the composition: thus, e.g., a high water content increases the tanning intensity; differences in the shade result depending on the pH value; liposomes as carrier act as tan intensifiers.

The intensity of the color is also dependent on the thickness of the horny layer of the epidermis. Very horny places, such as balls of the thumb and soles of the feet, are more strongly colored. The face takes on less color than the extremities, however, the individual differences are very great. Among 10 to 15% of all people the skin is not colored at all with DHA due to a particular skin constitution.

With certain endogenous products such as tryptophan and alanine, DHA leads to a more yellow dye; particularly with fair-skinned people with skin poorly supplied with blood, the skin often looks yellowish. Attempts have therefore been made to mix several substances (e.g., dihydroxyacetone with erythrulose; dihydroxyacetone with troxerutin (DHA Rapid, Merck), in order to obtain an attractive brown shade. Cosmetically attractive deep brown shades were achieved with a combination of dihydroxyacetone and mucon dialdehyde. The combination of mesotartaric dialdehyde with hydroquinone has also been proposed.

As it has been possible to show with histological sections, only the uppermost layers of the horny layer of the epidermis take on the shade. The tanning starts after about two to four hours, whereby a repetition of the application after two to three hours leads to optimal results. Depending on the speed of the shedding of the upper horny layers of the epidermis, the color change lasts three to seven days, then slowly fades, to disappear completely within five to fifteen days.

Self-tanning preparations with dihydroxyacetone or substances reacting in a chemically similar manner are absolutely harmless. Only the dead cell layer of the uppermost horny layers of the skin is dyed, damage to deeper layers of the skin is impossible. Since no side effects occur-the substances are not toxic and sensitizations are extremely rare there are no objections at all to a prolonged application of self-tanning preparations. However, the crucial disadvantage of self-tanning preparations containing DHA is that an application with self-tanners as the sole pigmentation carriers is satisfactory only in exceptional cases, since the resulting shade hardly corresponds to the color of natural healthy skin.

It would be desirable to provide self-tanning preparations (particularly emulsions) in a simple and cost-effective manner, which preparations are characterized in that the shade achieved with them is as close as possible to the shade of natural skin.

It would also be desirable to provide skin-protective preparations and preparations for skin care, whereby the preparations care for and improve the look of the appearance of the body. These preparations should thereby in particular also improve the moisture content of the skin.

### SUMMARY OF THE INVENTION

The present invention provides a cosmetic self-tanning composition which comprises dihydroxyacetone and one or more of a ginkgo extract, grapeseed oil and a lotus blossom extract.

In one aspect, the composition of the present invention may comprise at least a ginkgo extract. In another aspect, it may comprise at least grapeseed oil. In another aspect, it may comprise at least a lotus blossom extract. In another aspect, it may comprise at least a ginkgo extract and grapeseed oil. In another aspect, it may comprise a ginkgo extract, grapeseed oil and a lotus blossom extract.

In another aspect, the composition of the present invention may comprise from about 0.5% to about 10% by weight of dihydroxyacetone, based on the total weight of the composition. For example, the composition may comprise at least about 1% by weight of dihydroxyacetone and/or the composition may comprise not more than about 5% by weight of dihydroxyacetone.

In yet another aspect, the composition may comprise from about 0.001% to about 10% by weight of grapeseed oil, based on the total weight of the composition and/or the ginkgo extract may comprise one or more substances selected from flavone glucosides, bilobalide and ginkgolides.

In a still further aspect, the composition may comprise from 0 to less than about 5% by weight of UV filter substances, based on the total weight of the composition.

In another aspect, the composition may further comprise at least one of citronellol, citral, limonene, linalool, oxane, mandarin aldehyde, calone, cyclogalbanate, Florhydral, majantol, helional, cis-jasmone, caramel, coumarin, hexyl cinnamal, methyl cedryl ketone, 3-methyl-5-phenyl-1-pentanol and hexylsalicylate.

In another aspect, the composition may be substantially free of formaldehyde donors and/or parabens.

The present invention also provides an emulsion which comprises the composition of the present invention as set forth above, including the various aspects thereof. For example, the emulsion may be an O/W emulsion.

The present invention also provides a cosmetic self-tanning emulsion which comprises from about 0.5% to about 10% by weight of dihydroxyacetone, based on the total weight of the emulsion, and one or more of a ginkgo extract, grapeseed oil and a lotus blossom extract.

In one aspect, the emulsion may comprise a gingko extract and at least one of grapeseed oil and a lotus blossom extract.

In another aspect, the emulsion may be substantially free of parabens and/or formaldehyde donors.

In yet another aspect, the emulsion may comprise an O/W emulsion.

The present invention also provides a method of improving the elasticity the skin, the hair and/or the nails. The method comprises applying the composition of the present invention as set forth above, including the various aspects thereof, to the skin, the hair and/or the nails.

The present invention also provides a method of improving cellulite and/or “orange-peel skin”. The method comprises applying the composition of the present invention as set forth above, including the various aspects thereof, to the skin affected by cellulite and/or “orange-peel skin”.

### DETAILED DESCRIPTION OF THE PRESENT INVENTION

The preparations according to the present invention represent in every respect thoroughly satisfactory preparations, which are not limited to a restricted selection of raw materials. They show very good sensory and cosmetic properties, such as, e.g., dispersibility on the skin or absorbability into the skin with simultaneously excellent skin care data. The preparations according to the present invention are further characterized in that usually in particular the obtained shade of the skin is more natural than that achievable with comparable products, which contain neither a ginkgo extract nor grapeseed oil nor a lotus blossom extract.

It is therefore particularly advantageous for the purposes of the present invention, to use the cosmetic self-tanning preparations that contain dihydroxyacetone and further a ginkgo extract and/or grape-seed oil and/or a lotus blossom extract, to achieve a natural skin shade.

For the purposes of the present invention, “natural color tone” and “naturalness of the skin color tone” are understood as meaning that the tan obtained using self-tanning preparations is comparable with the skin tan or the skin color tone which is achieved by gentle suntanning. Whether a skin color tone should be referred to as natural can be determined, for example, by ascertaining the Natural Tanning Factor (NTF).

To describe a natural tan and determine the Natural Tanning Factor (NTF), reflectance spectra of the skin on the pale inside of the arm and on the suntanned outer arm are recorded using a standard commercial spectrometer by means of light guides.

The spectrum of the outer arm is divided in terms of wave length by the spectrum of the inside of the arm, i.e., standardized. For further evaluation, the standardized spectrum is used in each case.

If the standardized spectra for the untreated skin are now compared to those that were recorded 24 hours after the application of a self-tanning preparation, the more alike the shape and progress of the curves, the more natural the tan can be said to be.

The NTF corresponds to the ratio of the difference (as a measure of the slope in the spectra in these ranges) of the intensity values at 410 nm (Y₄₁₀) minus that at 500 nm (Y₅₀₀) and the difference in intensity values at 620 nm (Y₆₂₀) and 750 nm (Y₇₅₀):

NTF=(Y₄₁₀−Y₅₀₀)/(Y₆₂₀−Y₇₅₀)

Whether a self-tanning preparation produces a natural tan can thus be determined by a statistical test in which the individual NFTs of suntanned skin are compared to those of the skin of the same group of test subjects tanned with the aid of the self-tanning preparation. If there is no statistically relevant difference between the two data sets, the tans may be characterized as the same, and accordingly the artificial tan can be regarded as appearing natural.

It goes without saying that the evaluation of such a test can be made purely visually, e.g., by the test subjects themselves or with the aid of a different measuring method with which color impressions can be determined. In any case, however, it must be taken into account thereby that, as already stated, with 10 to 15% of all people the skin is not dyed at all with DHA. In this case, the test shown of course cannot be evaluated.

The application of preparations according to the invention, e.g., in the form of the cited examples, furthermore surprisingly leads to a considerable improvement in the appearance of the skin. In particular, through the application of preparations according to the invention:

the barrier properties of the skin are better retained or restored,

the drying-out of the skin is better counteracted and

the skin is better protected against environmental influences

The use of preparations according to the invention also increases the moisture content of the skin and the deep moisture content of the skin, this skin-moistening care effect and protection against moisture loss lasting for several hours or even over the whole day or even longer.

It is therefore furthermore particularly advantageous for the purposes of the present invention to use the cosmetic self-tanning compositions that contain dihydroxyacetone and further a ginkgo extract and/or grape-seed oil and/or a lotus blossom extract for increasing the skin moisture content or to moisten the skin.

The skin moisture content can be determined, for example, by using corneometry. The method is explained below by way of example in outline:
Acclimatization for at least 30 minutes under standard climate conditions (21.5±1° C. and 45±5% relative air humidity),
1^{st} measurement before the application,
One-time controlled application,
2^{nd} measurement two hours after the application.

The preparations according to the invention are moreover excellently suited for the care of sensitive skin.

It was furthermore surprising that through application of preparations according to the invention, the elasticity of the skin, the hair and/or the nails and therefore cellulite or the phenomenon of the so-called “orange-peel skin” may be improved.

The cosmetic composition according to the present invention preferably comprises at least about 0.5%, e.g., at least about 1% by weight, and not more than about 10%, e.g., not more than about 5% by weight of dihydroxyacetone, based on the total weight of the composition.

Ginkgo extract is an extract produced from the leaves of the “fossil” tree Ginkgo biloba native to Japan and China, which extract contains some or all of the following components: glucosides of flavones—especially quercetin—and isoquercetin glycoside, kaempferol-3-rhamnoside, luteolin glycoside and sitosterol glycoside—bilobalide and ginkgolides.

Particularly advantageous are ginkgo extracts that are obtained at room temperature with circulating white mineral oil (paraffin oil), whereby advantageously about 1 kg of extract is obtained from about 0.2 kg of dry material (10 kg of fresh plant corresponds to approx. 1 kg of dry material).

Ginkgo extracts from the company Botanica are particularly advantageous for the purposes of the present invention.

A cosmetic composition according to the present invention preferably contains from about 0.001% to about 5% by weight, preferably from about 0.01 % to about 3% by weight, particularly preferably from about 0.05% to about 1% by weight of a ginkgo extract, based on the total weight of the composition.

Grapeseed oil is the golden yellow to green, sweet/bitter tasting oil that is obtained from ground seeds of grapes (Vitis vinifera L., Vitaceae; oil content approx. 6% to 20% by weight) by pressing or extraction with hexane. Grape-seed oil contains approx. 95% of fatty acid glycerides and approx. 1% by weight of unsaponifiable matter, of which approx. half is accounted for by phytosterols. The triterpene erythrodiol (olean-12-en-3β,28-diol) is characteristic of grapeseed oil. The average fatty acid composition is: from about 65% to about 70% by weight of linoleic acid, from about 10% to about 20% by weight of oleic acid, from about 8% to about 10% by weight of palmitic acid, and from about 3% to about 5% by weight of stearic acid.

A cosmetic composition according to the present invention preferably comprises from about 0.001% to about 10% by weight, more preferably from about 0.01% to about 5% by weight, particularly preferably from about 0.1% to about 2% by weight of grapeseed oil, based on the total weight of the composition.

A lotus blossom extract which is suitable for the purposes of the present invention may be obtained, for example, by extraction from the blossom of botanically known lotus species (Nelumbo nucifera, Nelumbonaceae family), such as, e.g., Indian lotus (Nelumbo nucifera L.).

Particularly advantageous are lotus blossom extracts that are obtained at room temperature with a circulating glycerin/water mixture, whereby approx. 1 kg of extract is advantageously obtained from approx. 0.1 kg of dry material (10 kg of fresh plant corresponds to approx. 1 kg of dry material).

Typically, a lotus blossom extract for use in the present invention may comprise some or all of the following components: flavonoids (glucosides of quercitin, kaempferol) sterols, tannins, amino acids, essential oils.

Lotus blossom extracts which are commercially available from Botanica and Lotus Nelumbium Nelumbo HG obtainable from Alban Muller are particularly advantageous for the purposes of the present invention.

A cosmetic composition according to the present invention preferably comprises from about 0.001% to about 10% by weight, more preferably form about 0.01% to about 5% by weight, particularly preferably from about 0.1% to about 2% by weight of lotus blossom extract, based on the total weight of the composition.

It is advantageous for the purposes of the present invention to select the weight ratio of dihydroxyacetone to ginkgo extract and/or grape-seed oil and/or lotus blossom extract to be greater than about 1, in particularly advantageously from about 5:1 to about 10:1.

It is particularly preferred if the preparations according to the invention also comprise both a ginkgo extract and grapeseed oil in addition to dihydroxyacetone.

A property of cosmetic products that is very essential to the consumer, but difficult to measure objectively, is their scent or their smell. In a particularly preferred embodiment of the present invention, the cosmetic self-tanning composition comprises one or more substances selected from citronellol, citral, limonene, linalool, oxane, mandarin aldehyde, calone, cyclogalbanate, florhydral, majantol, helional, cis-jasmone, caramel, coumarin, hexyl cinnamal, methyl cedryl ketone, 3-methyl-5-phenyl-1-pentanol and hexyl salicylate.

Whereas dihydroxyacetone in cosmetic preparations of the prior art, in particular when it is a cosmetic containing water, usually decomposes over time, whereby degradation products (such as, e.g., hydroxylmethyl glyoxal, formaldehyde, glycolic acid, formic acid, acetic acid, pyroracemic acid) are formed, which in addition to coloring the preparation brown, lead to a reduction of the pH value of the same and to an unpleasant smell of the cosmetic composition, preparations according to the invention, which contain one or more of the cited substances, show very good sensory and cosmetic properties. The smell of the degradation products of the 1,3-dihydroxyacetone, usually perceived as pungent and intensive, which as a rule means the consumers no longer use the cosmetic, is considerably weakened or does not occur at all through the addition of the listed substances.

It is further preferred if the preparations according to the invention are emulsions, in particular O/W emulsions.

The cosmetic preparations according to the present invention may have a conventional composition and can be used to treat or care for the skin and as a cosmetic product in decorative cosmetics.

The oil phase of the preparations according to the present invention may comprise, for example, constituents which are advantageously selected from triglycerin esters of saturated or unsaturated, branched or unbranched alkanecarboxylic acids with a chain length of from about 8 to about 24, in particular from about 12 to about 18, carbon atoms. Caprylic/capric acid triglyceride (INCI: caprylic/capric triglyceride) may be used particularly advantageously for the purposes of the present invention.

The fatty acid triglycerides can advantageously be selected from synthetic, semisynthetic and natural oils, such as, e.g., olive oil, sunflower oil, soybean oil, peanut oil, rapeseed oil, almond oil, palm oil, coconut oil, castor oil, grapeseed oil, safflower oil, evening primrose oil, and the like.

For the purposes of the present invention, the oil phase may advantageously contain substances from the group of esters of saturated and/or unsaturated, branched and/or unbranched alkanecarboxylic acids with a chain length of from about 3 to about 30 carbon atoms and saturated and/or unsaturated, branched and/or unbranched alcohols with a chain length of from about 3 to about 30 carbon atoms, and from the group of esters of aromatic carboxylic acids and saturated and/or unsaturated, branched and/or unbranched alcohols with a chain length of from about 3 to about 30 carbon atoms. Such ester oils can then advantageously be chosen from the group of isopropyl palmitate, isopropyl stearate, n-butyl stearate, n-hexyl laurate, isooctyl stearate, isononyl stearate, isononyl isononanoate, 2-ethylhexyl palmitate, 2-hexyldecyl stearate, 2-octyidodecyl palmitate, erucyl erucate, and synthetic, semisynthetic and natural mixtures of such esters, such as, e.g. jojoba oil.

It is furthermore advantageous for the purposes of the present invention if the preparations contain one or more volatile lipids, e.g., from the group of phenyl trimethicone (INCI name: phenyl trimethicone, CAS 2116-84-9), cyclomethicone (INCI name cyclomethicone, CAS 556-67-2 and 69430-24-6), in particular octamethylcyclotetrasiloxane, cyclomethicone D5 and/or cylcomethicone D6, and C12-20 isoparaffin or isohexadecane.

The constituents of the oil phase can represent any mixtures of oil components, fat components and/or wax components.

It is advantageous for the purposes of the present invention if the preparations contain one or more glycerol esters, in particular glycerol esters of α-hydroxycarboxylic acids and saturated fatty acids, whereby the total amount of the glycerol esters in the finished cosmetic preparations is advantageously chosen from the range of from about 0.1% to about 10% by weight, preferably from about 0.5% to about 6% by weight, based on the total weight of the preparations.

Furthermore, it is advantageous for the purposes of the present invention, if the preparations contain one or more fatty alcohols. Cetyl alcohol is a preferred fatty alcohol.

The total amount of one or more fatty alcohols in the finished cosmetic preparations is advantageously chosen from the range of from about 0.1% to about 10% by weight, preferably from about 0.5% to about 6% by weight, based on the total weight of the preparations.

The cosmetic composition according to the present invention can advantageously be present in the form of a water-in-oil emulsion (W/O emulsion). In this case the following emulsifiers are preferred:

| Trade Name | INCI Name |
|---|---|
| Lameform TGI | Polyglyceryl-3 diisostearate |
| Isolan GI 34 | Polyglyceryl-4 isostearate |
| Dehymuls PGPH | Polyglyceryl-2 dipolyhydroxystearate |
| Arlacel P 135 | PEG-30 dipolyhydroxystearate |
| Eucerit PA | Lanolin alcohol |
| Atlas G-1049 | PEG-40 sorbitan perisostearate |
| Abil EM 90 | Cetyl dimethicone copolyol |
| Arlacel 989 | PEG-7 hydrogenated castor oil |
| Elfacos ST 9 | PEG 45/dodeceyl glycol copolymer |
| Elfacos ST 37 | PEG 22/dodeceyl glycol copolymer |
| Isostearinsaure PP | Pentaerythrityl isostearate |
| Imwitor 780 K | Isostearyl diglyceryl succinate |
| Arlacel 987 | Sorbitan isostearate |
| Hostacerin DGI | Polyglyceryl-2 sesquiisostearate |
| Tegin ISO | Glyderyl isostearate |
| Arlacel 60 | Sorbitan stearate |
| Tegin M | Glyceryl stearate |
| Arlantone G | PEG-25 hydrogenated castor oil |
| Arlantone T | PEG-40 sorbitan peroleate |
| Arlacel 80 | Sorbitan oleate |
| | Cera microcristallina + paraffinum |
| | liquidum + ozokerite + hydrogenated |
| | castor oil + glyceryl isostearate + polyglyceryl- |
| | 3 oleate |
| Atlas G-1049 | PEG-40 sorbitan perisostearate |
| ABIL WS 08 | Cetyl dimethicone copolyol + hexyl |
| | laurate + polyglyceryl-3 oleate + cetyl |
| | dimethicone |
| Hostacerin DGO | Polyglyceryl-2 sesquioleate |
| Abil WE 09 | Cetyl dimethicone copolyol (+) |
| | polyglyceryl-4 isostearate (+) hexyl |
| | laurate |
| Abil EM 90 | Cetyl PEG/PPG-10/1-dimethicone |
| Abil EM 97 | Dimethicone copolyol (+) |
| | cyclomethicone |
| Isolan GO 33 | Polyglyceryl-3 oleate |
| Montanov WO 18 | Isostearyl alcohol (+) isostearyl |
| | glucoside |
| Cremophor GO 32 | Polyglyceryl-3 dioleate |
| Olivem 900 | Sorbitan oleate |
| Sisterna SP01-C | Sucrose distearate |
| Sisterna SP 10-C | Sucrose distearate |
| Dehymuls B | Polyglyceryl-3 diisostearate (+) glyceryl |
| | oleate |
| Emulsogen SRH | Rapeseed sorbitols |
| Emulsogen SRO | Rapeseed sorbitols |
| Rylo PG 11 | Polyglyceryl dimer soyate |
| Grindstedt PS 401 | Polyglyceryl polyricinoleate |
| (Dermofeel PR) | |
| Isolan PDI | Polyglyceryl-3 dimerate |
| Arlacel 986 | Glyceryl sorbitan stearate |
| Decaglyn 5-HS | Polyglyceryl-10 hydroxystearate |

It is particularly preferred if the W/O emulsifier(s) is/are chosen from PEG-30 dipolyhydroxystearate, polyglyceryl-3-diisostearate, and PEG-40 sorbitan perisostearate.

In another preferred embodiment of the cosmetic composition of the present invention, the composition is present in the form of an oil-in-water emulsion (O/W emulsion). In this case, the O/W emulsifier(s) may advantageously be selected from glyceryl stearate in combination with ceteareth-20 and/or ceteareth-25, ceteareth-6 in combination with stearyl alcohol, cetylstearyl alcohol in combination with PEG-40 castor oil and sodium cetylstearyl sulfate, triceteareth-4 phosphate, glyceryl stearate, sodium cetylstearyl sulfate, lecithin trilaureth-4 phosphate, laureth-4 phosphate, stearic acid, propylene glycol stearate SE, PEG-25 hydrogenated castor oil, PEG-54 hydrogenated castor oil and/or PEG-6 caprylic acid/capric acid glyceride, glyceryl oleate in combination with propylene glycol, PEG-9 stearate, PEG-20 stearate, PEG-30 stearate, PEG40 stearate, PEG-100 stearate, ceteth-2, ceteth-20, polysorbate-20, polysorbate-60, polysorbate-65, and/or polysorbate-100, glyceryl stearate in combination with PEG-100 stearate, glyceryl myristate, glyceryl laurate, PEG-40 sorbitan peroleate, laureth-4, ceteareth-3 and/or isostearyl glyceryl ether, cetylstearyl alcohol in combination with sodium cetylstearyl sulfate, laureth-23 and/or steareth-2, glyceryl stearate in combination with PEG-30 stearate, PEG-40 stearate, glycol distearate, PEG-22 dodecyl glycol copolymer, polyglyceryl-2 PEG-4 stearate, ceteareth-12, ceteareth-20, ceteareth-30, methylglucose sesquistearate, steareth-10 and/or PEG-20 stearate, steareth-2 in combination with PEG-8 distearate, steareth-21, steareth-20, isosteareth-20, PEG-45/dodecyl glycol copolymer, methoxy-PEG-22/dodecyl glycol copolymer, PEG-40 sorbitan peroleate, PEG-40 sorbitan perisostearate, PEG-20 glyceryl stearate, PEG-20 glyceryl stearate, PEG-8 beeswax, polyglyceryl-2 laurate, isostearyl diglyceryl succinate, stearamidopropyl-PG dimoniumchloride phosphate, glyceryl stearate SE, ceteth-20, triethylcitrate, PEG-20 methylglucose sesquistearate, glyceryl stearate citrate, cetyl phosphate, cetearyl sulfate, sorbitan sesquioleate, triceteareth-4 phosphate, trilaureth-4 phosphate, polyglyceryl methylglucose distearate, potassium cetyl phosphate, polyglyceryl-3 methylglucose distearate isosteareth-10, polyglyceryl-2 sesquiisostearate, ceteth-10, oleth-20 and/or isoceteth-20, glyceryl stearate in combination with ceteareth-20, ceteareth-12, cetylstearyl alcohol and cetyl palmitate, cetylstearyl alcohol in combination with PEG-20 stearate, PEG-30 stearate, PEG-40 stearate and/or PEG-100 stearate.

According to the present invention, glyceryl stearate, PEG-40 stearate, triglycerin methylglucose distearate, polyglyceryl-3 methylglucose distearate, polyethylene glycol(21)stearyl ether, polyethylene glycol(2)stearyl ether, cetearyl glucoside, glyceryl stearate citrate, sodium cetearyl sulfate, cetearyl alcohol and/or sorbitan stearate may be used as particularly advantageous O/W emulsifiers.

The preparations according to the invention may be used in sufficient quantity on the skin in the customary manner for cosmetics.

The cosmetic preparations according to the present invention can comprise cosmetic auxiliaries as are customarily used in such preparations, e.g., preservatives, preservative aids, complexing agents, bactericides, perfumes, substances for preventing or increasing foaming, pigments that have a coloring effect, thickeners, moisturizing and/or humectant substances, fillers which improve the feel on the skin, and other customary constituents of a cosmetic composition, such as alcohols, polyols, polymers, foam stabilizers, electrolytes, organic solvents or silicone derivatives.

Suitable preservatives for the purposes of the present invention include, for example, formaldehyde donors (such as, for example, DMDM hydantoin, which is available, for example, under the trade name Glydant™ from Lonza), iodopropynyl butylcarbamates (e.g., those available under the trade names Glycacil-L, Glycacil-S from Lonza, and/or Dekaben LMB from Jan Dekker), parabens (i.e., alkyl p-hydroxybenzoates, such as methyl-, ethyl-, propyl- and/or butylparaben), phenoxyethanol, ethanol, benzoic acid and the like. In addition, the preservative system according to the present invention may also advantageously comprise preservative aids, such as, for example, octoxyglycerol, glycine soya, etc.

Although the listed preservatives per se can be advantageously used in the preparations according to the invention, those preparations are particularly preferred which are free of parabens and/or free of formaldehyde donors.

Advantageous complexing agents for the purposes of the present invention include, for example, EDTA, [S,S]-ethylenediamine disuccinate (EDDS), which is available, for example, under the trade name Octaquest from Octel, pentasodium ethylenediamine tetramethylenephosphonate, which is available, for example, under the trade name Dequest 2046 from Monsanto and/or iminodisuccinic acid, which is available, inter alia, from Bayer AG under the trade names Iminodisuccinate VP OC 370 (about 30% solution) and Baypure CX 100 solid.

Particularly advantageous preparations are also obtained if antioxidants are used as additives or active ingredients. According to the invention, the preparations advantageously comprise one or more antioxidants. Favorable, but nevertheless optional, antioxidants which may be used are all antioxidants which are customary or suitable for cosmetic applications.

For the purposes of the present invention, water-soluble antioxidants may be used particularly advantageously, such as, for example, vitamins, e.g. ascorbic acid and derivatives thereof.

Preferred antioxidants furthermore include vitamin E and derivatives thereof and vitamin A and derivatives thereof.

The amount of antioxidants (one or more compounds) in the preparations is preferably from about 0.001% to about 30% by weight, particularly preferably from about 0.05% to about 20% by weight, in particular from about 0.1% to about 10% by weight, based on the total weight of the preparation.

If vitamin E and/or derivatives thereof are the antioxidant or the antioxidants, it is advantageous to choose their respective concentrations from the range of from about 0.001% to about 10% by weight, based on the total weight of the composition.

If vitamin A or vitamin A derivatives, or carotenes or derivatives thereof are the antioxidant or the antioxidants, it is advantageous to choose their respective concentrations from the range of from about 0.001% to about 10% by weight, based on the total weight of the composition.

It is particularly advantageous if the cosmetic preparations according to the present invention comprise cosmetic active ingredients, preferred active ingredients being antioxidants which can protect the skin against oxidative stress.

Further advantageous active ingredients for the purpose of the present invention are natural active ingredients and/or derivatives thereof such as, e.g., alpha-lipoic acid, phytoen, D-biotin, Coenzyme Q10, alpha-glucosylrutin, carnitine, carnosine, natural and/or synthetic isoflavonoids, creatine, taurine and/or β-alanine, as well as 8-hexadecene-1,16-dicarboxylic acid (dioic acid, CAS No. 20701-68-2; provisional INCI designation octadecendioic acid).

Compositions according to the invention which comprise, for example, known antiwrinkle active ingredients, such as flavone glycosides (in particular α-glycosylrutin), coenzyme Q10, vitamin E and/or derivatives and the like are particularly advantageously suitable for the prophylaxis and treatment of cosmetic changes in the skin, as arise, for example, during skin ageing (such as, for example, dryness, roughness and formation of dryness wrinkles, itching, reduced regreasing (e.g., after washing), visible vascular dilations (telangiectasias, couperosis), flaccidity and formation of lines and wrinkles, local hyperpigmentation, hypopigmentation and incorrect pigmentation (e.g., age spots), increased susceptibility to mechanical stress (e.g., cracking) and the like). In addition, they are advantageously suitable for combating the appearance of dry and/or rough skin.

The water phase of the preparations according to the present invention can advantageously comprise customary cosmetic auxiliaries, such as, for example, alcohols, in particular those of low carbon number, preferably ethanol and isopropanol, polymers, foam stabilizers, electrolytes, and in particular one or more thickeners, which may advantageously be chosen from silicon dioxide, aluminum silicates, polysaccharides or derivatives thereof, e.g., hyaluronic acid, xanthan gum, hydroxypropylmethylcellulose, particularly advantageously from the group of polyacrylates, preferably a polyacrylate from the group of so-called carbopols [from Noveon], for example carbopol grades 980, 981, 1382, 2984, 5984, ETD 2020, ETD 2050, pemulen TR-1 or -2, Ultrez 10, in each case individually or in combination.

In addition, the preparations according to the present invention can advantageously also comprise repellents for protection against flies, ticks and spiders and the like. For example, N,N-diethyl-3-methylbenzamide (trade name: Meta-delphene, “DEET”), dimethyl phthalate (trade name: Palatinol M, DMP), and in particular ethyl 3-(N-n-butyl-N-acetylamino)propionate (available under the trade name Insekt Repellent® 3535 from Merck) are advantageous. Another advantageous repellent active ingredient for the purposes of the present invention is 1-piperidinecarboxylic acid 2-(2-hydroxyethyl)-1-methylpropylester (INN: Icaridin, CAS no. 119515-38-7, Elincs no.: 423-210-8) that has the following structure

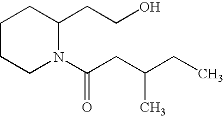

and is considered to be particularly effective.

The repellents can either be used individually or in combination.

Also so-called moisturizers can be advantageously used. Moisturizers is the term used to refer to substances or mixtures of substances which impart to cosmetic preparations the property, following application or distribution on the surface of the skin, of reducing moisture release by the horny layer (also called trans-epidermal water loss (TEWL)) and/or of positively influencing hydration of the horny layer.

For the purposes of the present invention, advantageous moisturizers include, e.g., glycerol, lactic acid and/or lactates, in particular sodium lactate, butylene glycol, propylene glycol, biosaccharide gum-1, glycine soya, ethylhexyloxyglycerol, pyrrolidonecarboxylic acid and urea.

The cosmetic preparations according to the present invention can also advantageously, but not necessarily, comprise fillers which, for example, further improve the sensory and cosmetic properties of the compositions and, for example, bring about or enhance a velvety or silky feel on the skin. Advantageous fillers for the purpose of the present invention include starch and starch derivatives (such as, for example, tapioca starch, distarch phosphate, aluminum or sodium starch octenylsuccinate and the like), pigments which have neither a primarily UV filter effect nor a coloring effect (such as, for example, boron nitride etc.) and/or Aerosils® (CAS No. 7631-86-9).

It is preferred according to the present invention to select the content of UV filter substances (one or more compounds) to be lower than about 5% by weight, in particular lower than about 2% by weight, based on the total weight of the preparation.

In the event that a content of UV filter substances is desired, these are advantageously selected from one or more of the following groups: UV A, UV B and/or broadband filter substances and organic or inorganic pigments as UV filter substances.

Particularly advantageous UV filter substances for the purposes of the present invention include:
Dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.
Phenylene-1,4-bis(2-benzimidazyl)-3,3′,5,5′-tetrasulfonic acid and its salts, particularly the corresponding sodium, potassium or triethanolammonium salts, in particular phenylene-1,4-bis(2-benzimidazyl)-3,3′,5,5′-tetrasulfonic acid bis-sodium salt with the INCI name Disodium Phenyl Dibenzimidazole Tetrasulfonate (CAS No. 180898-37-7), which is available, for example, under the trade name Neo Heliopan AP from Symrise;
Salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Symrise;
1,4-Di(2-oxo-10-sulfo-3-bornylidenemethyl)benzene (also: 3,3′-(1,4-phenylenedimethylene)bis(7,7-dimethyl-2-oxobicyclo[2.2.1]hept-1-ylmethanesulfonic acid) and salts thereof, (particularly the corresponding 10-sulfato compounds, in particular the corresponding sodium, potassium or triethanolammonium salt), that is also called benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;
Sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.
Benzoxazole derivatives, such as, for example, 2,4-bis[5-1(dimethylpropyl)benzoxazol-2-yl(4-phenyl)imino]-6-(2-ethylhexyl)imino-1,3,5-triazine with the CAS No. 288254-16-0, which is available from 3V Sigma under the trade name Uvasorb® K2A.

Hydroxybenzophenones, e.g. hexyl 2-(4′-diethylamino-2′-hydoxybenzoyl)benzoate (also: aminobenzophenone), which is available under the trade name Uvinul A Plus from BASF.
Triazine derivatives, such as, for example, 2,4-bis{[4-(2-ethylhexyloxy)-2-hydroxylphenyl}-6-(4-methoxyphenyl)-1,3,5-triazine (INCI: Bis-Ethylhexyloxy]phenol Methoxyphenyl Triazine), which is available under the trade name Tinosorb® S from CIBA-Chemikalien GmbH; dioctylbutylamidotriazone (INCI: Diethylhexyl Butamido Triazone), which is available under the trade name UVASORB HEB from Sigma 3V; tris(2-ethylhexyl) 4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150, 2-[4,6-bis(2,4-di methylphenyl)-1,3,5-triazin-2-yl]-5-(octyloxy)phenol (CAS No.: 2725-22-6).
Benzotriazoles, such as, for example, 2,2′-methylenebis(6-(2H-benzotriazol-2-yl)-4-(1,1,3,3-tetramethylbutyl)phenol) (INCI: Methylene Bis-Benztriazolyl Tetramethylbutylphenol), which is available, for example, under the trade name Tinosorb® M from CIBA-Chemikalien GmbH.
3-Benzylidenecamphor derivatives, preferably 3-(4-methylbenzylidene)camphor, 3-benzylidenecamphor;
4-Aminobenzoic acid derivatives, preferably 2-ethylhexyl 4-(dimethylamino)benzoate, amyl 4-(dimethylamino)benzoate;
Esters of benzalmalonic acid, preferably di(2-ethylhexyl) 4-methoxybenzalmalonate;
Esters of cinnamic acid, preferably 2-ethylhexyl 4-methoxycinnamate, isopentyl 4-methoxycinnamate;
Derivatives of benzophenone, preferably 2-hydroxy-4-methoxybenzophenone, 2-hydroxy-4-methoxy-4′-methylbenzophenone and 2,2′-dihydroxy-4-methoxybenzophenone; and
UV filters bonded to polymers;
ethylhexyl 2-cyano-3,3-diphenylacrylate (INCI: Octocrylene), which is available from BASF under the name Uvinul® N 539 T.

The following examples are intended to illustrate the present invention without restricting it. The numbers in the examples denote percent by weight, based on the total weight of the respective composition.

| W/O-Emulsions | 1 | 2 | 3 | 4 | 5 |
|---|---|---|---|---|---|
| Triglycerin diisostearate | 1.0 | 0.5 | 0.25 | 2.0 | 3.0 |
| Diglycerin dipolyhydroxystearate | 1.0 | 1.5 | 1.75 | 3.0 | 2.0 |
| Paraffin oil | 12.5 | 10.0 | 8.0 | 5.0 | 7.5 |
| Petrolatum | 8.0 | 6.0 | 5.0 | 2.0 | 2.5 |
| Hydrogenated cocoglycerides | 2.0 | 1.0 | 2.5 | 5.0 | 0.25 |
| Decyl oleate | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenates castor oil | 0.5 | 0.75 | 1.0 | 2.5 | 5.0 |
| Octyl salicylate | 0.5 | 1.0 | 1.5 | 2.0 | 3.5 |
| Dihydroxyacetone | 2.5 | 5.0 | 1.0 | 3.5 | 5.0 |
| Lotus blossom extract | 0.5 | — | — | — | 1.5 |
| Ginkgo extract | — | 1.5 | 0.1 | — | — |
| Grapeseed oil | — | — | — | 0.35 | — |
| Magnesium sulfate | 0.5 | 0.6 | 0.5 | 0.7 | 1.0 |
| Glycerin | 3.0 | 5.0 | 10.0 | 15.0 | 1.5 |
| Citric acid | 0.2 | 0.1 | 0.2 | 0.3 | 1.0 |
| Sodium citrate | 0.2 | 0.05 | 0.4 | 0.3 | 2.0 |
| Ethanol | 2.0 | — | 5.0 | — | — |
| Capric/caprylic acid triglyceride | 2.0 | 2.5 | 3.0 | 5.0 | 0.5 |
| Potassium sorbate | 0.04 | 0.15 | 0.05 | 0.03 | 0.4 |
| Benzyl alcohol | 0.3 | 0.4 | 0.25 | 0.15 | — |
| Water | ad | ad | ad | ad | ad |
| | 100 | 100 | 100 | 100 | 100 |
| W/O-Emulsions | 6 | 7 | 8 | 9 | 10 |
| PEG-30 Dipolyhydroxystearate | — | 0.5 | 0.25 | — | 3.0 |
| Lanolin Alcohol | 1.0 | 1.5 | 1.75 | 3.0 | — |
| Paraffin oil | 12.5 | 10.0 | 8.0 | 5.0 | 7.5 |
| Petrolatum | 8.0 | 6.0 | 5.0 | 4.0 | 2.5 |
| Hydrogenated cocoglycerides | 2.0 | 1.0 | 2.5 | 5.0 | 0.25 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Octyl salicylate | 0.5 | 1.0 | 1.5 | 2.0 | 3.5 |
| Dihydroxyacetone | 2.5 | 5.0 | 3.5 | 2.5 | 1.5 |
| Gingko extract | 0.25 | 1.5 | 0.35 | 1.0 | 0.5 |
| Magnesium sulfate | 0.5 | 0.6 | 0.5 | 0.7 | 1.0 |
| Glycerin | 3.0 | 5.0 | 10.0 | 15.0 | 1.5 |
| Citric acid | 0.2 | 0.1 | 0.2 | 0.3 | 1.0 |
| 1,3-Butylene glycol | 2.0 | — | 5.0 | — | — |
| Capric/caprylic acid triglyceride | 2.0 | 2.5 | 3.0 | 5.0 | 0.5 |
| Sodium dehydracet | — | — | 0.05 | — | — |
| Ptoassium sorbate | 0.3 | 0.4 | 0.25 | 0.15 | — |
| Talc | — | — | 0.05 | — | 0.1 |
| Water | ad | ad | ad | ad | ad |
| | 100 | 100 | 100 | 100 | 100 |
| W/S (Water-in-Silicone)- | | | | | |
| Emulsions | 11 | 12 | 13 | 14 | 15 |
| Cetyl PEG/PPG-10/1 Dimethicone | 1.0 | — | — | 3.0 | 5.0 |
| Cylomethicone + PEG/PPG-18/18 | 10.0 | 12.5 | 25 | — | — |
| Dimethicone (90:10) | | | | | |
| Cyclomethicone | 12.5 | 15 | 28.0 | 25.0 | 17.5 |
| Dimethicone | 5.0 | 13.0 | 5.0 | 12.0 | 15.0 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerin | 3.0 | 5.0 | 10.0 | 15.0 | 1.5 |
| Ginkgo extract | 0.5 | 1.0 | 0.5 | 0.7 | 1.0 |
| Dihydroxyacetone | 2.5 | 5.0 | 1.5 | 3.5 | 2.0 |
| Citric acid | 0.2 | 0.1 | 0.2 | 0.3 | 1.0 |
| Sodium citrate | 1.0 | 0.1 | 0.4 | 0.9 | 2.5 |
| Potassium sorbate | 0.4 | 0.1 | 0.05 | 0.3 | 0.4 |
| Cetyldimethicone | 0.5 | — | 0.7 | — | — |
| Benzyl alcohol | — | — | 0.05 | — | 0.1 |
| Water | ad | ad | ad | ad | ad |
| | 100 | 100 | 100 | 100 | 100 |
| W/S-Emulsions | 16 | 17 | 18 | 19 | 20 |
| Cetyl PEG/PPG-10/1 Dimethicone | 1.0 | — | — | 3.0 | 5.0 |
| Cylomethicone + PEG/PPG-18/18 | 10.0 | 12.5 | 25 | — | — |
| Dimethicone (90:10) | | | | | |
| Cyclomethicone | 12.5 | 15 | 18.0 | 15.0 | 11.5 |
| Dimethicone | 5.0 | 13.0 | 5.0 | 12.0 | 5.0 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerin | 3.0 | 5.0 | 10.0 | 15.0 | 1.5 |
| Lactic acid | 0.2 | 0.1 | 0.2 | — | — |
| Sodium lactate | 0.2 | 1.0 | 0.05 | — | — |
| Grapeseed oil | 0.5 | 1.0 | 1.5 | 0.5 | 0.1 |
| Dihydroxyacetone | 2.5 | 5.0 | 3.5 | 2.0 | 1.0 |
| Stearyldimethicone | 0.5 | — | 0.7 | — | — |
| Dehydracetic acid | — | — | 0.05 | — | 0.1 |
| Modified starch | — | 2.5 | — | 0.15 | — |
| Water | ad | ad | ad | ad | ad |
| | 100 | 100 | 100 | 100 | 100 |
| W/O-Emulsions | 21 | 22 | 23 | 24 | 25 |
| PEG-22 Dodecyl Glycol | 5.0 | 1.5 | 0.25 | — | 3.0 |
| Copolymer | | | | | |
| PEG-45 Dodecyl Glycol Polymer | 1.0 | 1.5 | 1.75 | 3.0 | — |
| Paraffin oil | 12.5 | 10.0 | 8.0 | 5.0 | 17.5 |
| Isopropyl stearate | 8.0 | 6.0 | 5.0 | 12.0 | 2.5 |
| Hydrogenated cocoglycerides | 2.0 | 1.0 | 2.5 | 5.0 | 0.25 |
| Evening primrose oil | 0.5 | 0.75 | 1.0 | 2.0 | 0.25 |
| Octyldodecanol | 0.5 | 1.0 | 0.75 | 3.0 | 0.25 |
| Aluminum stearat | 0.4 | 0.3 | 0.6 | 1.0 | 0.05 |
| Dicaprylyl carbonate | 0.1 | 0.05 | 0.15 | 0.5 | 1.0 |
| Hydorgenated castor oil | 0.5 | 0.75 | 1.0 | 2.5 | 5.0 |
| Ginkgo extract | 0.5 | 1.0 | 1.5 | 0.2 | 0.1 |
| Dihydroxyacetone | 2.5 | 5.0 | 3.5 | 2.0 | 1.0 |
| Magnesium sulfate | 0.5 | 0.6 | 0.5 | 0.7 | 1.0 |
| Glycerin | 3.0 | 5.0 | 10.0 | 15.0 | 1.5 |
| Sodium citrate | 0.2 | 0.1 | — | — | — |
| Citric acid | 0.2 | 0.1 | — | — | — |
| 1,3-Butylene glycol | 2.0 | — | 5.0 | — | — |
| Capric/caprylic acid triglyceride | 2.0 | 2.5 | 3.0 | 5.0 | 0.5 |
| Potassium sorbate | 0.4 | 0.15 | 0.05 | 0.3 | 0.4 |
| Benzyl alcohol | — | — | 0.05 | — | 0.1 |
| Water | ad | ad | ad | ad | ad |
| | 100 | 100 | 100 | 100 | 100 |
| W/O-Emulsions | 26 | 27 | 28 | 29 | 30 |
| Polyglyceryl-2- | 3.0 | — | 0.25 | — | 3.0 |
| dipolyhydroxystearate | | | | | |
| Polyglyceryl-3 diisostearate | 1.0 | 3.5 | 1.75 | 2.5 | — |
| PEG-40 sorbitan isostearate | — | 2.5 | 0.5 | 3.5 | 3.0 |
| Paraffin oil | 12.5 | 10.0 | 8.0 | 5.0 | 17.5 |
| Isopropyl stearate | 8.0 | 6.0 | 5.0 | 12.0 | 2.5 |
| Hydrogenated cocoglycerides | 2.0 | 1.0 | 2.5 | 5.0 | 0.25 |
| Isopropyl palmitate | 0.5 | 1.0 | 0.75 | 3.0 | 0.25 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Grapeseed oil | 0.5 | 1.0 | 1.5 | 0.25 | 0.1 |
| Dihydroxyacetone | 2.5 | 5.0 | 3.5 | 2.5 | 1.0 |
| Magnesium sulfate | 0.5 | 0.6 | 0.5 | 0.7 | 1.0 |
| Glycerin | 3.0 | 5.0 | 10.0 | 15.0 | 1.5 |
| Citric acid | 0.2 | 0.1 | 0.1 | 0.3 | 1.0 |
| Sodium citrate | 0.2 | 0.3 | 0.2 | 1.5 | 0.8 |
| Capric/caprylic acid triglyceride | 2.0 | 2.5 | 3.0 | 5.0 | 0.5 |
| Potassium sorbate | 0.24 | 0.15 | 0.05 | 0.3 | 0.4 |
| Water | ad | ad | ad | ad | ad |
| | 100 | 100 | 100 | 100 | 100 |
| Silicone-in-Water-Emulsions | 31 | 32 | 33 | 34 | 35 |
| Dimethicone copolyol, Caprylic/ | 1.0 | 2.0 | 8.0 | 3.0 | 5.0 |
| Capric triglyceride | | | | | |
| Cyclomethicone | 12.5 | 15 | 25.0 | 10.0 | 7.5 |
| Dimethicone | 5.0 | 15.0 | 5.0 | 12.0 | 15.0 |
| Mineral oil | 0.5 | 0.75 | 1.0 | 2.0 | 0.25 |
| Phenyltrimethicone | 0.5 | 1.0 | 0.75 | 3.0 | 0.25 |
| Glycerin | 5.0 | 7.5 | 10.0 | 3.0 | 1.0 |
| Xanthan Gum | — | 0.1 | — | 0.25 | 1.0 |
| Lotus blossom extract | 0.5 | — | — | — | 1.5 |
| Ginkgo extract | — | 1.5 | 0.1 | — | — |
| Grapeseed oil | — | — | — | 0.35 | — |
| Panthenol | 0.5 | 1.0 | 0.75 | 0.25 | 0.1 |
| Methyl paraben | 0.4 | 0.1 | 0.05 | 0.3 | 0.4 |
| Octylsalicylat | 0.5 | 1.0 | 1.5 | 2.0 | 3.5 |
| Dihydroxyacetone | 2.5 | 5.0 | 3.5 | 2.0 | 1.0 |
| Propyl paraben | 0.3 | 0.4 | 0.25 | 0.15 | — |
| Iodopropynyl butylcarbamate | — | — | 0.05 | — | 0.1 |
| Water | ad | ad | ad | ad | ad |
| | 100 | 100 | 100 | 100 | 100 |
| O/W-Emulsions | 36 | 37 | 38 | 39 | 40 |
| Glyceryl stearate | 1.0 | — | — | 0.5 | 0.25 |
| Polyethylenglycol(40) stearate | 10.0 | — | 5 | — | — |
| Triglycerin methylglucose | — | 4.0 | — | — | 2.5 |
| distearate | | | | | |
| Myristyl myristate | 1.0 | 2.0 | 0.5 | — | 1.0 |
| Vitamin E acetate | 0.5 | 1.0 | 0.25 | 0.1 | — |
| Citric acid | — | 0.1 | 3.0 | — | — |
| Cyclomethicone | 2.5 | 5.0 | 8.0 | 5.0 | 7.5 |
| Octyl methoxycinnamate | 5.0 | 7.5 | 3.0 | — | — |
| Xanthan gum | 0.5 | 0.3 | 0.1 | 0.05 | 1.0 |
| Dimethicone | 5.0 | 1.0 | 5.0 | 2.0 | 5.0 |
| Butylmethoxy dibenzoylmethane | 1.0 | 2.0 | 3.0 | — | — |
| Cetyl alcohol | 1.0 | 2.0 | 2.0 | 1.0 | — |
| Sodium EDTA | 0.2 | 0.2 | 0.4 | — | — |
| Octyl salicylate | 0.5 | 3.0 | 1.5 | 2.0 | 3.5 |
| Grapeseed oil | 0.5 | 0.5 | 0.1 | 0.25 | 0.35 |
| Dihydroxyaceton | 2.5 | 2.0 | 2.0 | 1.0 | 2.5 |
| Methylpropandiole | 0.5 | 2.0 | 1.0 | 2.0 | 0.25 |
| Phenoxyethanol | 0.5 | 0.4 | 0.75 | 3.0 | 0.25 |
| Methyl paraben | 0.4 | 0.2 | 0.05 | 0.3 | 0.4 |
| Propyl paraben | 0.3 | 0.2 | 0.25 | 0.15 | — |
| Perfume | q.s. | q.s. | q.s. | q.s. | q.s. |
| Glycerin | 5.0 | 8.0 | 3.0 | 15.0 | 7.5 |
| Tapioka starch | — | 1.5 | — | 0.15 | — |
| Water | ad | ad | ad | ad | ad |
| | 100 | 100 | 100 | 100 | 100 |
| O/W-Emulsions | 41 | 42 | 43 | 44 | 45 |
| Glyceryl stearate citrate | 1.0 | — | — | 0.5 | 4.5 |
| Glyceryl monostearate | 1.0 | 1.0 | 5.0 | — | — |
| Triglycerin methylglucose | — | — | — | 5.0 | — |
| distearate | | | | | |
| Cetearyl alcohol + sodium | 5.0 | 3.0 | — | — | — |
| cetearylsulfate | | | | | |
| Dimethicone | 0.5 | 0.5 | 0.75 | 1.5 | 0.2 |
| Behenyl alcohol | 1 | — | 2 | 1 | 0.2 |
| Ginkgo extract | 0.3 | 0.25 | 0.1 | 1.0 | 0.15 |
| Stearyl alcohol | — | — | — | 1 | 0.2 |
| Cetylstearyl alcohol | — | — | 1 | 1 | 0.2 |
| Xanthan Gum | 0.5 | 0.3 | 0.75 | 0.25 | 0.1 |
| Octyldodecanol | 0.5 | 3.0 | 0.75 | 3.0 | 0.25 |
| C12–15 Alkyl benzoate | 0.5 | 1.0 | 0.75 | 0.25 | 0.1 |
| Octyl salicylate | 0.5 | 0.5 | 1.5 | 2.0 | 3.5 |
| Dihydroxyacetone | 2.5 | 2.5 | 3.5 | 2.0 | 1.5 |
| Grapeseed oil | 0.05 | 0.5 | 0.15 | 0.1 | — |
| Caprylic/Capric Triglycerid | 1.0 | 2.0 | 3.0 | 5.0 | 10 |
| Methyl paraben | 0.4 | 0.3 | 0.05 | 0.3 | 0.4 |
| Propyl paraben | 0.3 | 0.1 | 0.25 | 0.15 | — |
| Ethanol | — | 3.5 | 1.5 | — | 1.0 |
| Phenoxyethanol | — | 0.5 | — | 0.15 | — |
| C13–16 Isoparaffin | 10 | 3.0 | — | 5.0 | — |
| Citric acid | — | 0.09 | — | 5 | 10 |
| Sodium citrate | — | 0.175 | 10 | 5 | — |
| Glycerin | 5.0 | 8.0 | 3.5 | 15 | 10 |
| Water | ad | ad | ad | ad | ad |
| | 100 | 100 | 100 | 100 | 100 |
| Gels | 46 | 47 | 48 | 49 | 50 |
| Polyacrylic acid, Na salt | 1.0 | 0.2 | 0.5 | 0.1 | 1.5 |
| PEG-40 stearate | 0.5 | — | 0.75 | — | — |
| Carrageenan | 1.25 | 0.2 | 0.2 | 0.1 | 2.5 |
| Sodium polyacrylate | 0.2 | 0.2 | 0.3 | 0.5 | 1.5 |
| Vinylpyrrolidone/Vinylacetat- | 1.0 | — | 0.2 | — | — |
| Copolymers | | | | | |
| Acrylates/C10–C30 Alkyl Acrylate | 1.5 | 0.2 | 0.15 | 1.5 | 0.75 |
| Crosspolymer | | | | | |
| Water-soluble color | 0.01 | 0.4 | — | — | — |
| Cyclomethicone | 1 | 8.5 | 5 | 0.5 | 3 |
| Dimethiconol | 0.5 | 0.5 | 0.75 | 1.25 | 1.0 |
| Cetyl stearyl alcohol | — | — | 1 | 1 | — |
| Hydrogenated polyisobutene | 0.5 | — | 1.0 | — | 0.25 |
| Lotus blossom extract | 0.05 | 1.0 | 0.1 | 0.5 | 0.35 |
| Dihydroxyacetone | 0.25 | 5.0 | 1.0 | 2.5 | 3.5 |
| Panthenol | 0.5 | — | 0.75 | 0.25 | 0.1 |
| Glycerin | 3.0 | 8.6 | 12.5 | 17.2 | 5.0 |
| Perfume | q.s. | q.s. | q.s. | q.s. | q.s. |
| Methylpropandiol | 4.5 | 2.0 | 0.5 | 1.5 | — |
| Propyl paraben | 0.3 | — | 0.25 | 0.15 | — |
| Ethanol | — | 10.0 | 5.0 | — | 3.5 |
| Water | ad | ad | ad | ad | ad |
| | 100 | 100 | 100 | 100 | 100 |

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A cosmetic self-tanning composition which comprises dihydroxyacetone and one or more of a ginkgo extract, grapeseed oil and a lotus blossom extract.

2. The composition of claim 1, wherein the composition comprises a ginkgo extract.

3. The composition of claim 1, wherein the composition comprises grapeseed oil.

4. The composition of claim 1, wherein the composition comprises a lotus blossom extract.

5. The composition of claim 1, wherein the composition comprises a ginkgo extract and grapeseed oil.

6. The composition of claim 1, wherein the composition comprises a ginkgo extract, grapeseed oil and a lotus blossom extract.

7. The composition of claim 1, wherein the composition comprises from about 0.5% to about 10% by weight of dihydroxyacetone, based on a total weight of the composition.

8. The composition of claim 7, wherein the composition comprises at least about 1% by weight of dihydroxyacetone.

9. The composition of claim 8, wherein the composition comprises not more than about 5% by weight of dihydroxyacetone.

10. The composition of claim 3, wherein the composition comprises from about 0.001% to about 10% by weight of grapeseed oil, based on a total weight of the composition.

11. The composition of claim 2, wherein the ginkgo extract comprises one or more substances selected from flavone glucosides, bilobalide and ginkgolides.

12. The composition of claim 1, wherein the composition comprises from 0 to less than about 5% by weight of UV filter substances, based on a total weight of the composition.

13. The composition of claim 1, wherein the composition further comprises one or more of citronellol, citral, limonene, linalool, oxane, mandarin aldehyde, calone, cyclogalbanate, florhydral, majantol, helional, cis-jasmone, caramel, coumarin, hexyl cinnamal, methyl cedryl ketone, 3-methyl-5-phenyl-1-pentanol and hexylsalicylate.

14. The composition of claim 1, wherein the composition is substantially free of formaldehyde donors.

15. The composition of claim 1, wherein the composition is substantially free of parabens.

16. An emulsion which comprises the composition of claim 1.

17. The emulsion of claim 16, which comprises an O/W emulsion.

18. A cosmetic self-tanning emulsion which comprises from about 0.5% to about 10% by weight of dihydroxyacetone, based on a total weight of the emulsion, and one or more of a ginkgo extract, grapeseed oil and a lotus blossom extract.

19. The emulsion of claim 18, wherein the emulsion comprises a gingko extract and at least one of grapeseed oil and a lotus blossom extract.

20. The emulsion of claim 19, wherein the emulsion is substantially free of parabens and formaldehyde donors.

21. The emulsion of claim 18, wherein the emulsion comprises an O/W emulsion.

22. A method of improving the elasticity of at least one of skin, hair and nails, wherein the method comprises applying to at least one of skin, hair and nails the composition of claim 1.

23. A method of treating cellulite or “orange-peel skin”, wherein the method comprises applying to affected skin the composition of claim 1.
